(19) United States
(12) Patent Application Publication
(10) Pub. No.: US 2012/0178913 A1
(43) Pub. Date: Jul. 12, 2012

(54) OLEFIN METATHESIS REACTIONS OF AMINO ACIDS, PEPTIDES AND PROTEINS CONTAINING ALLYL SULFIDE GROUPS

(75) Inventors: Yuya Angel Lin, Oxford (GB); Justin Mark Chalker, Oxford (GB); Benjamin Guy Davis, Oxford (GB)

(73) Assignee: ISIS INNOVATION LIMITED, Oxford (GB)

(21) Appl. No.: 12/996,237

(22) PCT Filed: Jun. 5, 2009
(86) PCT No.: PCT/GB2009/001429

§ 371 Date: Mar. 3, 2011

(30) Foreign Application Priority Data
Jun. 6, 2008 (GB) 0810418.4

(51) Int. Cl. C07C 319/18; C07K 1/00

(52) U.S. Cl. 530/410; 560/153; 530/395

(57) ABSTRACT

A method for the modification of an amino acid, protein or peptide is disclosed. The method comprises reacting a carbon-carbon double bond-containing compound with an amino acid, a protein or a peptide containing an allyl sulfide group in the presence of a catalyst which promotes olefin metathesis, to form a modified amino acid, protein or peptide. Preferred carbon-carbon double bond-containing compounds include carbohydrates.

The present application is concerned with methods for the modification of proteins via olefin metathesis reactions.

Post-translational modifications of naturally occurring proteins, such as glycosylation or methylation, are of importance in many biological processes such as cell signalling and regulation, development and immunity. Site-selective chemical modification of protein surfaces is of interest for the study and possible modification or control of protein function.

The introduction of a double bond into the side chain of an amino acid in a peptide or protein creates a non-natural chemical functionality that may be further selectively modified by olefin metathesis chemistry to link that side chain, and hence the peptide or protein, to additional groups including carbohydrates. By controlling the site at which the double bond is introduced and the functionality that is used in the linking chemistry, site-selective modification of peptides or proteins can be achieved. WO2005/000873 exemplifies the use of olefin cross-metathesis reactions to modify the side chains of both individual amino acids and short peptide chains, but does not exemplify the actual modification of any proteins. There is therefore a need for further methods that allow the rapid and efficient preparation of a wide range of modified proteins under mild conditions.

In one aspect, the present invention therefore provides a method for the modification of an amino acid, protein or peptide, the method comprising reacting a carbon-carbon double bond-containing compound I with an amino acid, a protein or a peptide containing an allyl sulfide group II in the presence of a catalyst which promotes olefin metathesis, to form a modified amino acid, protein or peptide III. Preferably, the reaction takes place in an aqueous solvent.

It has surprisingly been found that allyl sulfide groups react rapidly and efficiently in olefin cross-metathesis reactions. Self-metathesis of alkene I may occur if it is an allylsulfide itself or if it is allyl alcohol. In general, however, this does not occur for other alkenes I or is not a problem since secondary metathesis events can put self-metathesis products back into the catalytic cycle. The reaction is rapid enough that, for many starting materials II, it can be carried out in aqueous solvents in the open air and at ambient temperature before catalyst decomposition and loss of activity occurs. The fact that the metathesis reaction is rapid is particularly surprising in view of a number of reports in the literature of sulfur-containing substrates chelating to the metal ion in the metathesis catalyst, which sequesters the catalyst in an unproductive form and hence prevents metathesis (see for example A. Fürstner et al, Tetrahedron 55 (1999) 8215-8230; and J. L. Mascareñas et al, J. Org. Chem., 1997, 62, 8620-8621).

As used herein, an allyl sulfide group comprises a sulfide group attached to a carbon-carbon double bond via one intervening carbon atom, that is a group with the core structure C═C—C—S—. It has been found that replacement of the S atom in the allyl sulfide with an O atom or an NH group leads to much lower yields of the desired product. Increasing the number of carbon atoms between the S atom and the C═C bond also leads to much lower yields of the desired product. For these reason, the presence of an allyl sulfide group in compound II is an essential feature of the methods of the present invention.

The allyl sulfide group is present in the side chain of an amino acid. The amino acid is preferably an a-amino acid. It may optionally be incorporated into a peptide or protein. The amino acid may be in the D- or L-form, preferably the L-form. As used herein, a peptide contains a minimum of two amino acid residues linked together via an amide bond.

Preferred carbon-carbon double bond-containing compounds I, preferred amino acids, proteins and peptides containing an allyl sulfide group II, and preferred modified amino acids, proteins and peptides III are those shown in Scheme 1 below.

A preferred embodiment of the method of the invention is illustrated in Scheme 1.

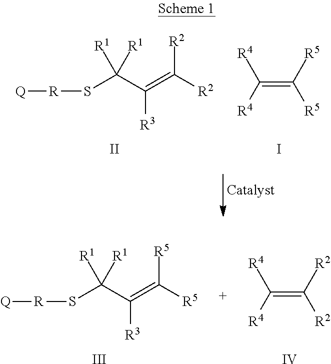

wherein

R denotes an amino acid side chain or a linker;

Q is an amino acid, peptide or protein;

each R¹ independently denotes H or C₁₋₁₀ alkyl;

each R² independently denotes H or C₁₋₁₀ alkyl;

R³ denotes H or C₁₋₁₀ alkyl;

each R⁴ independently denotes H or C₁₋₁₀ alkyl;

each R⁵ independently denotes H or any organic moiety it is desired to introduce into an amino acid, peptide or protein;

The invention includes any and all possible combinations of any preferred features referred to herein, whether or not such combinations are specifically disclosed.

There is no real limitation on the nature of the group R, as long as the S-allyl moiety is able to take part in the cross-metathesis reaction. The R group is attached to the backbone of the protein or peptide if Q denotes a peptide or protein. If Q denotes a protein, the allyl sulfide group must be on or near the surface of the protein such that the reaction can occur. Preferred R groups are those wherein the S atom it attached to a carbon atom in the group R, including alkylene groups and aryl groups. Preferred R groups include —CH₂— and —CH₂CH₂—.

Q is preferably a protein.

Preferred R¹ groups are independently H or C₁₋₄ alkyl, more preferably H or methyl and most preferably both R¹ denote H.

Preferred R² groups are independently H or C₁₋₄ alkyl, more preferably H or methyl and most preferably both R² denote H.

Preferably R³ denotes H or methyl, most preferably H.

Preferred R⁴ groups are independently H or C₁₋₄ alkyl, more preferably H or methyl and most preferably both R⁴ denote H.

Preferred methods include those wherein one R⁵ denotes H and the other denotes an organic moiety. Preferably, at least one of the R⁵ groups is a carbohydrate moiety, a polyethylene glycol (PEG) chain, a farnesyl group, a label or a pharmaceutically active compound.

As used herein, alkyl preferably denotes a straight chain or branched saturated hydrocarbon group containing 1-10 carbon atoms, preferably 1-6 carbon atoms and more preferably 1 to 4 carbon atoms. Straight chain alkyl groups are preferred. In the above definitions, any alkyl groups may be optionally substituted with any functional groups which do not interfere with the reaction of compounds I and II.

As used herein, aryl preferably denotes phenyl.

In principle, the alkene I can be any alkene which is reactive in an olefin metathesis reaction (for a review of reactivity of olefins in such reactions see Grubbs et al, J. Am. Chem. Soc., 2003, 125, 11360-11370, which is hereby incorporated by reference). Preferred are alkenes I which are soluble in the reaction solvent. Terminal alkenes are also preferred. The alkene may optionally be functionalised with any functional groups which do not interfere with the metathesis reaction. To avoid possible mixtures of products, it is also preferred that the alkene I contains a single C═C.

Preferably, an excess of the alkene I is used, for example from about 2 to about 20,000 mol equivalents, preferably between 4 and 10 mol equivalents, based on compound II, to help drive the reaction towards the desired product III.

Suitable alkenes I include allyl alcohols such as CH₂═CH—CH₂—OH, allyl amines, other allyl sulfides, farnesene and derivatives thereof, and C═C functionalised carbohydrate moieties. Suitable carbohydrate moieties include double bond containing derivatives of monosaccharides, oligosaccharides and polysaccharides, including derivatives of any carbohydrate moiety which is present in a naturally occurring glycoprotein or in biological systems. Preferred are glycosyl or glycoside derivatives, for example glucosyl or galactosyl derivatives. Glycosyl and glycoside groups include both α and β groups. Particularly preferred are C-glycosides, due to their potential hydrolytic stability (i.e. resistance to hydrolysis).

Suitable carbohydrate moieties include carbon-carbon double bond containing derivatives of glucose, galactose, fucose, GlcNAc, GalNAc, sialic acid, and mannose, or oligosaccharides or polysaccharides comprising at least one glucose, galactose, fucose, GlcNAc, GalNAc, sialic acid, and/or mannose residue. Preferred are alpha and beta vinyl and allyl C-glycosides of all of the above sugars, and oligosaccharides containing such C-glycoside derivatives. Particularly preferred are allyl C-glycosides.

Particularly preferred carbohydrate moieties include allyl glycosides, including allyl-α-D-galactopyranoside, allyl-β-D-galactopyranoside, allyl-α-D-glucopyranoside, allyl-β-D-glucopyranoside, allyl-α-D-mannopyranoside and allyl-β-D-mannopyranoside, and glycosylalkenes including 3-(α-D-galactopyranosyl)propene, 3-(β-D-galactopyranosyl)propene, 3-(α-D-glucopyranosyl)propene and 3-(β-D-glucopyranosyl)propene.

Preferably, any saccharide units making up the carbohydrate moiety which are derived from naturally occurring sugars will each be in the naturally occurring enantiomeric form, which may be either the D-form (e.g. D-glucose or D-galactose), or the L-form (e.g. L-rhamnose or L-fucose). Any anomeric linkages may be α- or β-linkages.

Carbon-carbon double bond containing carbohydrate compounds may be prepared by methods known in the art for the derivatisation of carbohydrates, in particular known methods for the formation of glycosylalkenes and alkenyl glycosides (see for example D. E. Levy & C. Tang, “Chemistry of the C-Glycosides”, 1995, and WO2005/000873, the disclosures of which are hereby incorporated by reference). Some such compounds are also commercially available. Suitable carbon-carbon double bond containing carbohydrate compounds based on D-glucose are shown below:

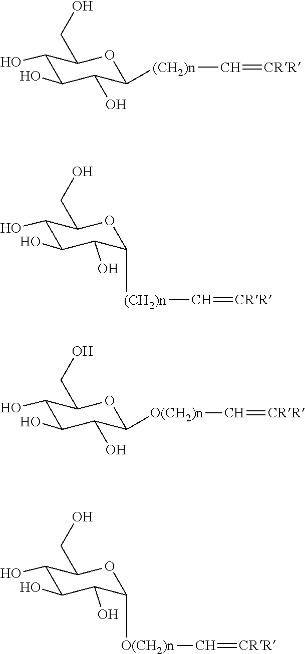

wherein n denotes 1 or 2; and

each R′ independently denotes hydrogen or an organic moiety, for example an alkyl group (e.g. a C₁₋₁₀ alkyl group). Each of the OH groups may be replaced by an —O-saccharide group.

Other suitable alkenes I include labels such as dyes, affinity tags (e.g. biotin), fluorescent tags or radio labelled compounds containing a C═C bond. Reaction with such compounds allows introduction of a suitable label into an amino acid, peptide or protein.

Other functionalised alkenes can also be used as alkene I to introduce additional functionality into an amino acid, peptide or protein. For example, a carbon-carbon double bond-containing amino acid can be used as the alkene I to add another amino acid residue to the side chain of a peptide or protein. The methods of the invention also allow pharmaceutically active compounds such as drugs or vaccines to be attached to an amino acid, peptide or protein via a C═C linkage.

Alkenes substituted with a PEG chain may also be used as compound I. Preferred are compounds wherein a PEG chain is attached to a terminal carbon-carbon double bond, such as CH₂═CH—CH₂—O—(CH₂CH₂O)n-H or CH₂═CH—CH₂—O—(CH₂CH₂O)n-CH₃, wherein n denotes an integer of 2 or more, for example 2 to 1000.

The olefin metathesis reaction is an equilibrium reaction. The driving force for the reaction is the liberation of the alkene IV which displaces the equilibrium towards the desired product. Preferably, the alkene IV is a gaseous alkene such as ethene which is readily removed from the reaction mixture, so helping drive the reaction towards the desired product.

The olefin metathesis reaction takes place in the presence of a catalyst. Suitable catalysts include those known in the art for such reactions, including complexes of tungsten, molybdenum, rhenium and ruthenium, preferably ruthenium. The catalyst should be selected to be compatible with any other functional groups present in the starting materials I and II. Preferred are catalysts that are at least partially soluble in water at the temperature of the reaction. A particularly preferred catalyst is the Grubbs-Hoveyda 2nd generation catalyst V shown below:

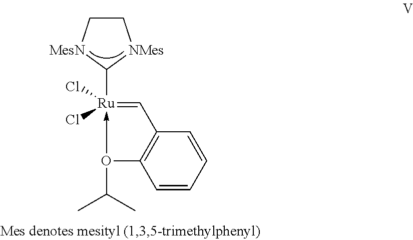

This catalyst, and other suitable catalysts, are commercially available. The catalyst can be used in any suitable amount, for example about 2-500 mol %, preferably about 2-10 mol %, based on the starting material II.

The olefin metathesis reaction can be carried out in any solvent in which the allyl sulfide group in compound II is available for reaction. Preferably, the starting material II is soluble in the solvent. For proteins, preferred solvents are aqueous solvents, for example aqueous buffers. The solvent chosen should be compatible with the protein starting material II and product III. Aqueous buffers which maintain the pH during the reaction at a value which will not cause significant damage to the proteins involved are preferred. Low amounts of tert-butanol may also be used to aid in solubilising the catalyst and/or the alkene I. Other suitable solvents include alcohols including methanol, ethanol and isopropanol (IPA), tetrahydrofuran (THF), dioxane, dimethoxyethane (DME), and diglyme. When the starting material II is an amino acid or peptide, organic solvents such as dimethylformamide (DMF), dimethylsulfoxide (DMSO), dichloromethane, dichloroethane and dioxane may also be used. The solvent should not however complex to the catalyst as this may prevent the reaction occurring. For this reason, solvent systems containing large amounts of DMSO are not considered to be suitable.

The olefin methathesis catalysts tend to decompose in aqueous environments. However, it has now been found that the cross-methathesis reaction of the allyl sulfides II is fast enough to favourably compete with catalyst decomposition in aqueous systems, thus allowing the reaction to be carried out in aqueous systems and open to the air.

Preferred reaction temperatures for proteins are in the range of about 4-37° C., to avoid unnecessarily damaging the protein starting material or product. Peptides and amino acids can be reacted at higher or lower temperatures (e.g. about 0-100° C.) if desired as they are generally less susceptible to temperature-induced damage. One of the advantages of the method of the invention is that it may be carried out under mild conditions which minimise the risk of damage to any proteins. The use of aqueous solvent systems and the fact that no precautions need to be taken to exclude air from the reaction mean that the method of the reaction is relatively cheap and easy to carry out on a commercially useful scale.

If the starting material II contains functional groups which may complex to the catalyst and hence hinder the metathesis reaction, then an oxophilic metal salt can be added to the reaction mixture to disrupt any such non-productive complexation. Suitable metal salts include Mg²⁺, Ca²⁺ and Zn²⁺ salts, preferably Mg²⁺ salts. The anion are not critical, as long as they are compatible with the starting materials and products under the reaction conditions. Suitable anions include chloride, bromide, sulfate and BF₄⁻. Preferably, the salt is soluble in the reaction medium. Sufficient salt should be added to disrupt the interaction of all the relevant functional groups in the starting material with the catalyst. Preferably, an excess of salt will be used, based on the relevant functional groups in the starting material II.

Other functional groups in the starting materials I and II may also be protected during the reaction using protecting groups known in the art (see for example Theodora W. Greene and Peter G. M. Wuts, Protective Groups in Organic Synthesis, 3rd Edition, 1999, John Wiley & Sons, Inc.).

The product III produced according to the methods of the invention comprises a moiety linked to the amino acid, peptide or protein via a linker which contains a carbon-carbon double bond. The carbon-carbon double bond may be cis or trans. The stereochemistry about the double bond may be influenced by choice of a suitable catalyst and reaction conditions for the olefin metathesis reaction. Bulky substituents on the starting materials will tend to favour a trans configuration around the double bond in the product III. The C═C bond in the product can be reduced if desired, for example by catalytic hydrogenation.

Preferred proteins for use as starting materials II in the methods according to the invention include enzymes, the selectivity of which may be modified by controlled glycosylation using the methods according to the invention, and therapeutic proteins. Other preferred proteins include serum albumins and other blood proteins, hormones, interferons, receptors, antibodies, interleukins and erythropoietin.

The methods of the invention allow access to derivatives which may be used to investigate or control protein function in vivo. For example, the methods of the invention allow labelling of proteins with markers which may be useful in monitoring protein function. Using the methods of the invention, proteins may also be modified to act as carriers for drugs or other pharmaceutically active molecules in vivo.

An allyl sulfide group may be introduced into an amino acid, peptide or protein by any method known in the art. For example, an amino acid comprising an allyl sulfide group in the side chain may be incorporated into a protein or peptide structure using standard methods for the preparation of peptides and proteins.

The thiol group in the side chain of cysteine may be converted into an allyl sulfide group via conversion of the cysteine to dehydroalanine and subsequent trapping with a suitable thiol nucleophile. For example, reaction of O-mesitlyenesulfonylhydroxylamine (MSH) with cysteine rapidly generates dehydroalanine which can be trapped with a thiol nucleophile such as allyl thiol or allyl methyl sulfide to yield the corresponding allyl sulfide derivative. The reaction may be carried out on cysteine itself, or on a cysteine residue which is present in a peptide or protein (Bernardes, G. J. L.; Chalker, J. M.; Errey, J. C.; Davis, B. G. J. Am. Chem. Soc., 2008, 130, pages 5052-3, which is hereby incorporated by reference). S-alkyl cysteines can also be converted to allyl sulfides using the same chemistry. This method may produce a mixture of epimers when the thiol nucleophile adds to the dehydroalanine double bond. This may or may not be a disadvantage depending on the intended use of the product III.

In an alternative electrophilic process, cysteine may converted directly into S-allyl cysteine by reaction with a suitable allylic halide compound. One advantage of this method is that it produces only one diastereomer of the S-allyl cysteine product.

The direct allylation of cysteine itself and of cysteine-containing peptides is known in the art (M. J. Brown, et al, J. Am. Chem. Soc., 1991, 113, 3176-3177; K. Kuhn, et al, J. Am. Chem. Soc., 2001, 123, 1023-1035; B. Ludolph, et al, J. Am. Chem. Soc., 2002, 124, 5954-5955). For example, cysteine in which the amino and acid functionalities have been protected can be allylated directly by reaction with allyl chloride in DMF at room temperature. It has now been found that direct allylation of cysteine residues on the surface of a protein is also possible. Thus, direct allylation of cysteine residues in a protein may be achieved by treating a buffered aqueous solution of the protein with a solution of an allyl halide in a suitable solvent, for example DMF. Suitable allyl halides include allyl chloride, allyl bromide and allyl iodide, with allyl chloride being preferred. The solvent should be chosen such that homogeneity is achieved when the solution of the allyl halide reagent is added to the protein solution. The amount and type of solvent for the allyl halide should also be chosen to minimise the risk of it causing any damage to the protein. For example, if DMF is used the reaction can take place even if the total amount of DMF does not exceed 5% of the total volume of the buffer, and most proteins can tolerate this level of DMF. Selective allylation of cysteine residues in the presence of other amino acids generally occurs even if a large excess of the allyl halide is used. If the cysteine residue in a protein is prone to oxidation, then it may be pre-reduced using dithiothreitol prior to treatment with the allyl halide.

US 2007/0260041, the disclosure of which is hereby incorporated by reference, discloses a method for the preparation of allylic sulfides which involves an allylic disulfide rearrangement followed by desulfurization. This method is suitable for the conversion of thiol groups in amino acids (e.g. cysteine) and peptides to allylic sulfide groups, for use in the methods of the invention.

A further alternative electrophilic method, which is specific for cysteine and hence is particularly suitable for use on proteins, involves the formation and dechalcogenative rearrangement of allylic selenosulfides. Such reactions are known in the art for cysteine itself and for cysteine-containing peptides (D. Crich, et al, J. Am. Chem. Soc., 2006, 128, 2544-2545; D. Crich, et al, J. Am. Chem. Soc., 2007, 129, 10282-10294, the disclosures of which are hereby incorporated by reference). Cysteine residues can be converted into the corresponding Se-allyl-selenosulfide by treatment with suitable allylic seleno-reagents, as exemplified in Scheme 2 below wherein X denotes a leaving group such as CN, SO₂Aryl (where Ar=e.g. phenyl or 4-methylphenyl), SO₂R (R=e.g. alkyl), SO₃⁻, I, Br, Cl, or OH, preferably CN, and Q denotes a protein or peptide.

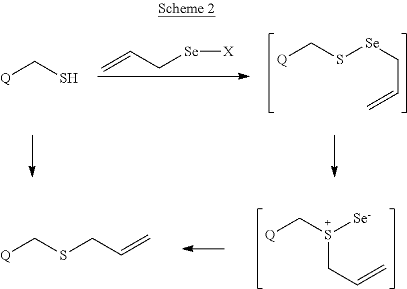

The reaction proceeds via formation of an Se-allyl selenosulfide followed by spontaneous loss of selenium to give the corresponding S-allyl cysteine. The reaction can be carried out at room temperature in an aqueous buffer, and thus provides a mild and efficient process for introducing an allyl sulfide group into a cysteine-containing protein without denaturing the protein. The method also has the advantage that only a single diastereomer of S-allyl cysteine is produced.

A peptide or protein may naturally contain one or more cysteine residues. Alternatively, a cysteine containing peptide or protein may be prepared via site-directed mutagenesis to introduce a cysteine residue at a desired position. Site-directed mutagenesis is a known technique in the art (see for example WO00/01712 and J. Sambrook et al, Molecular Cloning: A Laboratory Manual, 3rd Edition, Cold Springs Harbour Laboratory Press, 2001, the disclosures of which are hereby incorporated by reference).

S-allyl cysteine (Sac) may also be incorporated into proteins by methionine auxotropic E. Coli such as the B834 strain. In this strategy, Sac is used as a surrogate for methionine in methionine depleted media, with the expressed proteins at least partially incorporating Sac in place of methionine. A similar strategy has been used in the art to introduce other analogues of methionine into proteins (Jan C. M. van Hest, Kristi L. Kiick and David A. Tirrell, J. Am. Chem. Soc., 2000, 122, pages 1282-1288). Other genetic techniques for the introduction of non-natural amino acids into proteins are known in the art and could be adapted to provide suitable starting materials II for use in the methods of the invention (see for example J. Xie and P. Schultz, Nature Reviews: Molecular Cell Biology, 7, 2006, 775-782; and L. Wang and P. Shultz, Angew. Chem. Int. Ed., 2005, 44, 34-66).

The product III may be purified by any suitable method known in the art. In particular, protein products may be separated from any catalyst residues and/or product IV by affinity chromatography, size exclusion chromatography or dialysis. Ruthenium-based catalysts will complex to DMSO and can therefore be removed by washing with or dialysing against DMSO or a DMSO-containing buffer.

It is possible to envisage an analogous method to the method of the invention in which an allyl sulfide compound is reacted with an amino acid, peptide or protein containing an carbon-carbon double bond. However, it is chemically simpler and more efficient to introduce an allyl sulfide group into an amino acid, peptide or protein and then react it with an alkene, rather than introducing an alkene group into the amino acid, peptide or protein and then reacting it with an allyl sulfide compound. In particular, the method of the invention can utilise an excess of more cheaply and readily available starting material I in order to help drive the reaction to the desired product III.

The invention will be further illustrated by the following non-limiting Examples.

### EXAMPLES

The following abbreviations are used in the Examples: MeOH=methanol; Et₂O=diethyl ether; EtOAc=ethyl acetate; DMF=dimethylformamide; iPrOH=isopropanol; PBu₃=tributylphosphine; Et₃N=triethylamine; Boc=tert-butoxycarbonyl; aq.=aqueous; sat.=saturated; TLC=thin layer chromatography; TFA=trifluoroacetic acid; THF=tetrahydrofuran; brsm=based on recovered starting material; TBS=tert-butyldimethylsilyl; TBAF=tetrabutylammonium fluoride.

### Example 1

### N-Boc-L-Cysteine methyl ester (BocCysOMe)

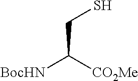

Anhydrous MeOH (100 mL) was added to a flame dried 250 mL round bottom flask equipped with a Teflon coated stir bar. The solvent was stirred and cooled to 0° C. and acetyl chloride (248 mmol, 17.6 mL) was added dropwise over 5 minutes. The solution was stirred an additional 10 minutes at 0° C. to give a concentrated solution of HCl. L-Cysteine (2.00 g, 16.51 mmol) was then added in one portion and the flask flushed briefly with argon. The ice bath was removed and the reaction was stirred at room temperature for 24 hours. The solvent was then removed under reduced pressure to give the crude cysteine methyl ester hydrochloride as a pale yellow solid. This material was used immediately in the next step without purification. The crude ester was suspended in CH₂Cl₂ (100 mL) and cooled to 0° C. Et₃N (5.06 mL, 36.3 mmol) was added carefully followed by di-tert-butyl dicarbonate (Boc₂O, 4.32 g, 19.81 mmol). The reaction was stirred at room temperature for 3.25 hours after which time TLC (30% EtOAc in Petrol) revealed the desired product (Rf=0.6) and its corresponding disulfide (Rf=0.3). The solvent was removed under reduced pressure and the resulting residue was redissolved in MeOH (40 mL) and H₂O (8 mL). Tributylphosphine (2.0 mL, 8.1 mmol) was added dropwise to the stirred solution. TLC revealed reduction of the disulfide. The reaction was diluted with Et₂O (100 mL) and H₂O (50 mL). The organic layer was separated and the aqueous layer was extracted with Et₂O (2×50 mL). The combined organics were washed with brine (100 mL), dried over MgSO₄, and filtered. The solvent was removed under reduced pressure and the residue purified by column chromatography eluting first with 5% EtOAc in petrol and then 20% EtOAc in petrol. The title compound was isolated as a clear oil (3.48 g, 89% from L-cysteine). ¹H NMR (400 MHz, CDCl₃): δ_{H}=1.42 (10H, s, includes Boc and SH), 2.94 (2H, app. td, J=4.3, 8.7 Hz, CH₂SH), 3.76 (3H, s, CO₂CH₃), 4.58 (1H, m, Hα), 5.44 (1H, d, J=5.8 Hz, NH).

If PBu₃ was not used to reduce disulfide, the reaction mixture could be purified by column chromatography to give BocCysOMe and the corresponding disulfide.

### Example 2

### N-Boc-S-allyl-cysteine methyl ester (BocSacOMe)

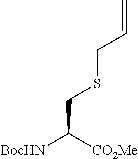

BocCysOMe (16.75 g, 71.17 mmol) was added to a 250 mL round bottom flask and placed under an atmosphere of argon before dissolving in DMF (71 mL). K2CO3 (24.59 g, 178 mmol) was added to the stirred solution and the mixture was cooled to 0° C. Allyl chloride (27.54 mL, 356 mmol) was added by syringe, the ice bath was removed, and the reaction was stirred vigorously at room temperature for 15 hours. TLC (25% EtOAc in petrol) indicated complete consumption of BocCysOMe (Rf=0.5) and formation of the allylated product (Rf=0.6). The reaction was diluted with 400 mL each of Et2O and H2O and separated. The organic layer was washed with H2O (2×200 mL) and then brine (2×200 mL). The organic layer was dried (MgSO4), filtered, and concentrated under reduced pressure. The product was purified by column chromatography (15% EtOAc in petrol) yielding 18.05 g as a clear oil (92%) which solidified to waxy prisms upon storage at −20° C. m.p.=37-38° C. 1H NMR (400 MHz, CDCl3): δH=1.46 (9H, s, Boc), 2.83-2.96 (2H, ABX System, J=14.0, 4.8, 5.5 Hz, CH2SAllyl), 3.09-3.19 (2H, m, SCH2CH═CH2), 3.77 (3H, s, CO2CH3), 4.53 (1H, m, Hα), 5.11-5.15 (2H, m, HC═CH₂), 5.33 (1H, d, J=6.8 Hz, NH), 5.76 (1H, m, HC═CH₂).

### Example 3

### N-Acetyl-L-cysteine methyl ester (AcCysOMe)

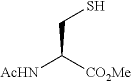

Thionyl chloride (SOCl₂, 10.0 mL, 137.9 mmol) was added dropwise to a stirred solution of N-acetyl-L-cysteine (15.0 g, 91.9 mmol) in anhydrous methanol (140 mL) under argon at 0° C. The reaction mixture was allowed to warm slowly to room temperature and then stirred for 6 hours. The solvent was removed under reduced pressure and the resulting residue diluted with EtOAc (250 mL) and water (250 mL). The organic layer was separated, and the aqueous layer was further extracted with EtOAc (250 mL). The combined organic layers were washed with brine (2×150 mL) and then dried over MgSO₄ and filtered. The solvent was removed under reduced pressure to give a thick, colourless oil that solidified on standing at −20° C. overnight. This material was used without purification. ¹H NMR (400 MHz, CDCl₃): δ_{H}=1.35 (1H, t, J=9.0 Hz, SH), 2.07 (3H, s, CH₃CO), 3.01 (2H, ddd, J=9.0, 4.0, 2.4 Hz, CH₂SH), 3.79 (3H, s, CO₂CH₃), 4.89 (1H, ddd, J=7.7, 4.0, 3.9 Hz, H_{α}), 6.45 (1H, br s, NH).

### Example 4

### N-Acetyl-S-allyl-L-cysteine methyl ester (AcSacOMe)

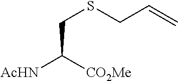

Allyl chloride (1.00 mL, 12.41 mmol) was added to a stirred DMF solution (40 mL) containing AcCysOMe (2.00 g, 11.28 mmol) and K₂CO₃ (1.87 g, 13.54 mmol) at room temperature. The reaction was monitored by TLC until no starting material was observed. Excess allyl chloride was removed under reduced pressure and the crude material was used in subsequent steps without purification (1.32 g, 54%). Spectroscopic data was consistent with that previously reported (Crich, D.; Krishnamurthy, V.; Brebion, F.; Karatholuvhu, M.; Subramanian, V.; Hutton, T. K. J. Am. Chem. Soc. 2007, 129, 10282-10294).

### Example 5

### Cross-Metathesis of BocSacOMe and Allyl Alcohol

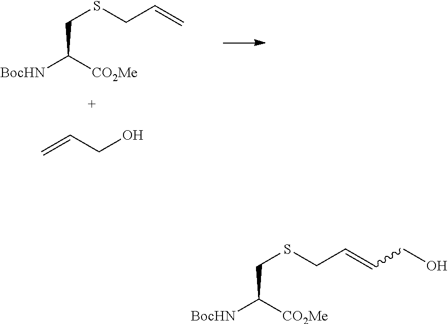

A solution of Hoveyda-Grubbs 2nd generation catalyst V (12 mg, 0.02 mmol) in tBuOH (2.0 mL) was added to a stirred mixture containing BocSacOMe (126 mg, 0.46 mmol) and allyl alcohol (0.16 mL, 2.29 mmol) in tBuOH:H2O (1:2, 6 mL) at 32° C. A second dose of the catalyst solution (12 mg in 2.0 mL tBuOH) and allyl alcohol (0.16 mL, 2.29 mmol) followed by 2.0 mL of water was added one hour later. After an additional 1.5 hours of reaction time, the brown mixture was concentrated under reduced pressure to yield a dark brown residue. Purification by column chromatography (50% EtOAc in petrol) afforded the starting material (30 mg, 24%) and cross-metathesis product (78 mg, 56%, 74% brsm). 1H NMR (400 MHz, CDCl3): δH=1.43 (9H, s, Boc), 2.49 (1H, br s, OH), 2.76-2.94 (2H, ABX System, J=13.8, 5.0, 5.8, Hβ), 3.14 (2H, d, J=7.1 Hz, SCH2CH═), 3.75 (3H, s, CO2CH3), 4.11 (2H, d, J=3.8 Hz, CH2OH), 4.41-4.56 (1H, m, Hα), 5.35 (1H, d, J=7.8 Hz, NH), 5.58-5.70 (1H, m, SCH2CH═), 5.70-5.83 (1H, m, ═CHCH₂OH).

### Example 6

### N-Boc-DL-Homocysteine methyl ester (BocHomoCysOMe)

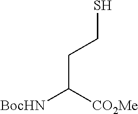

DL-homocysteine (2.00 g, 16.51 mmol) was added to a flame dried 250 mL round bottom flask equipped with a Teflon coated stir bar with the flask flushed with argon briefly. Anhydrous MeOH (160 mL) was then added via a syringe. The mixture was stirred and cooled to 0° C. and thionyl chloride (55.70 mmol, 4.0 mL) was added dropwise over 5 minutes. The ice bath was removed and the reaction was stirred at room temperature for 24 hours. The solvent was then removed under reduced pressure to give the crude cysteine methyl ester hydrochloride and the corresponding disulfide as a thick yellow oil. This material was used immediately in the next step without purification. The crude ester was suspended in CH₂Cl₂ (160 mL) and cooled to 0° C. Et₃N (10.35 mL, 74.27 mmol) was added carefully followed by di-tert-butyl dicarbonate (Boc₂O, 8.90 g, 40.85 mmol). The reaction was stirred at room temperature for 1.5 hours. The solvent was removed under reduced pressure and the resulting residue was redissolved in MeOH (80 mL) and H₂O (35 mL). Tributylphosphine (9.16 mL, 37.14 mmol) was added dropwise to the stirred solution. The reaction was diluted with Et₂O (200 mL) and H₂O (100 mL). The organic layer was separated and the aqueous layer was extracted with Et₂O (2×100 mL). The combined organics were washed with brine (200 mL), dried over MgSO₄, and filtered. The solvent was removed under reduced pressure and the residue purified by column chromatography eluting first with 5% EtOAc in petrol and then 20% EtOAc in petrol. The title compound was isolated as a clear oil (2.52 g, 27% from DL-homocysteine). 1H NMR (400 MHz, CDCl₃) δH=1.44 (9H, s, Boc), 1.57 (1H, t, J=8.45 Hz, SH) 1.85-2.01 (1H, m, Hβ), 2.05-2.18 (1H, m, Hβ′), 2.46-2.70 (2H, m, CH₂SAllyl), 3.75 (3H, s, COCH₃), 4.39-4.54 (1H, m, Hα), 5.10 (1H, d, J=7.00 Hz, NH).

### Example 7

### N-Boc-S-allyl-DL-homocysteine methyl ester (BocAhcOMe)

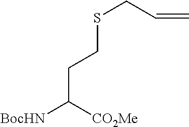

DMF (30 mL) was added to a 250 mL round bottom flask containing BocHomoCysOMe (1.97 g, 7.91 mmol). K2CO3 (1.64 g, 11.86 mmol) was added to the stirred solution. Allyl chloride (1.29 mL, 15.81 mmol) was added by syringe in two portions and the reaction was stirred vigorously at room temperature for 1 hour. A second dose of allyl chloride (1.29 mL, 15.81 mmol) was added by syringe and the reaction was stirred vigorously at room temperature for further 2 hours. The reaction was diluted with 100 mL each of Et20 and H2O, Et2O layer was separated. The aqueous layer was further extracted with Et2O (2×80 mL). The combined organic layer was washed with brine (100 mL). The organic layer was dried (MgSO4), filtered, and concentrated under reduced pressure. The product was purified by column chromatography (10% EtOAc in petrol then 20% EtOAc in petrol) yielding 2.06 g of the title compound as a clear oil (90%) which solidified to waxy prisms upon storage at −20° C. 1H NMR (400 MHz, CDCl3) δH=1.44 (9H, s, Boc), 1.82-1.97 (1H, m, Hβ), 2.01-2.16 (1H, m, Hβ), 2.40-2.56 (2H, m, CH2SAllyl), 3.12 (2H, d, J=7.3 Hz, CH₂CH═CH₂), 3.75 (3H, s, CO₂CH₃), 4.34-4.47 (1H, m, Hα), 5.03-5.12 (2H, m, CH═CH₂), 5.68-5.83 (1H, m, CH═CH₂).

### Example 8

### Cross-Metathesis of BocAhcOMe and Allyl Alcohol

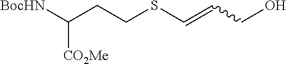

A solution of Hoveyda-Grubbs 2nd generation catalyst (7.6 mg, 0.02 mmol) in tBuOH (1.0 ml) was added to a stirred mixture containing BocAhcOMe (87.9 mg, 0.30 mmol) and allyl alcohol (0.10 ml, 1.52 mmol) in tBuOH:H2O (1:1.5, 5 ml) at 32° C. A second dose of the catalyst solution (7.6 mg 1 in 1.0 ml tBuOH) and allyl alcohol (0.10 ml, 1.52 mmol) followed by 1.0 ml of water was added one hour later. After an additional 1.5 hours of reaction time, the brown mixture was concentrated under reduced pressure to yield a dark brown residue. Purification by column chromatography (50% EtOAc in petrol) afforded the starting material (29 mg, 33%) and cross-metathesis product (65 mg, 67%, 99% brsm). 1H NMR (400 MHz, CDCl3) δH=1.42 (9H, s, Boc), 1.80-1.94 (1H, m, Hβ), 2.05 (1H, td, J=13.4, 7.3 Hz, Hβ), 2.38 (1H, br. s, OH), 2.48 (2H, t, J=7.8 Hz, CH2SCH2CH═), 3.11 (2H, d, J=6.3 Hz, SCH₂CH═CH), 3.73 (3 H, s, CO₂CH₃), 4.11 (2H, d, J=4.8 Hz, CH₂OH), 4.28-4.43 (1H, m, Hα), 5.26 (1H, d, J=7.8 Hz, NH), 5.57-5.77 (2H, m, CH═CH).

### Example 9

### Allyl2,3,4,6-tetra-O-acetyl-β-(D)-glucopyranoside

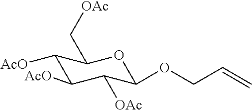

β-(D)-glucose pentaacetate (10.00 g, 25.60 mmol) was added to a 250 mL flame dried round bottom flask under argon and dissolved in CH₂Cl₂ (60 mL). The stirred solution was cooled to 0° C. and BF₃.OEt₂ (4.87 mL, 38.43 mmol) was added by syringe. After stirring 10 minutes at 0° C., allyl alcohol (2.61 mL, 38.43 mmol) was added. The ice bath was removed after completion of the addition and the reaction stirred at room temperature for 8.5 hours. The reaction was then cooled to 0° C. and quenched with NaHCO₃ (sat., aq, 50 mL). After dilution with H₂O (50 mL), the organic layer was separated and the aqueous layer was extracted with CH₂Cl₂ (2×100 mL). The combined organic layers were washed with brine (100 mL), dried over MgSO₄, filtered, and concentrated under reduced pressure. The product was purified by column chromatography (40% EtOAc in petrol) to give the title compound as a bright white solid (6.14 g, 62%). m.p.=73-75° C. This material was spectroscopically identical to that previously reported (Rodriguez, E. B.; Stick, R. V. Aust. J. Chem. 1990, 43, 665-679).

### Example 10

### Allyl-β-(D)-glucopyranoside

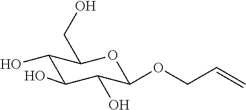

To a stirred solution of allyl 2,3,4,6-tetra-O-acetyl-β-(D)-glucopyranoside (2.00 g, 5.15 mmol) in MeOH (20 ml) was added sodium methoxide (139 mg, 0.26 mmol). After stirring for 20 minutes, the reaction was neutralized with Dowex 50WX8 (H⁺ form) and then filtered. The resulting solution was concentrated under reduced pressure to give a thick oil. Purification by column chromatography (30% ⁱPrOH in EtOAc) afforded the title compound as a thick clear oil which crystallized on standing (1.10 g, 97%). m.p.=96-98° C. Spectroscopic data was consistent with that previously reported (Kishida, M.; Akita, H. Tetrahedron 2005, 61, 10559-10568).

### Example 11

### Allyl 2,3,4,6-tetra-O-acetyl-α-(D)-mannopyranoside

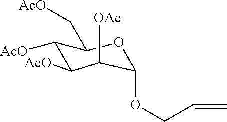

α-(D)-Mannose pentaacetate (3.50 g, 8.97 mmol) was added to a 100 mL 2-neck round-bottom flask and dissolved in CH₂Cl₂ (50 mL) and placed under nitrogen. The stirred solution was cooled to 0° C. and BF₃.OEt₂ (1.36 mL, 10.80 mmol) was added followed by allyl alcohol (0.92 mL, 13.46 mmol). The reaction was warmed to room temperature over several hours. After 12 hours, the reaction was quenched at 0° C. with NaHCO₃ (10 mL, sat., aq.) and diluted with H₂O (100 mL). The organic layer was separated and the aqueous layer was extracted with CH₂Cl₂ (2×100 mL). The combined organics were dried (MgSO₄), filtered, and concentrated under reduced pressure. Column chromatography (50% EtOAc in petrol) afforded the title compound as a clear oil (348 mg, 10%). ¹H NMR (400 MHz, CDCl₃): δ_{H}=1.90, 1.96, 2.02, 2.07 (3H, s, 4×OAc), 3.90-3.97 (2H, m, contains H5 and CHHCH═CH₂), 4.01 (1H, dd, J=12.3, 2.4, H6), 4.08-4.14 (1H, m, CHHCH═CH₂), 4.20 (1H, dd, J=12.3, 5.3, H6′), 4.78 (1H, dd, J=1.8, H1), 5.13-5.22 (3H, m, contains H2 and CH═CH₂), 5.24-5.29 (2H, m, H3 and H4), 5.76-5.86 (1H, m, CH═CH₂).

### Example 12

### Allyl-α-(D)-mannopyranoside

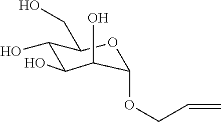

The product from Example 11 (260 mg, 0.67 mmol) was added to a 25 mL round bottom flask and dissolved in 10 mL MeOH. To the stirred solution at room temperature was added NaOMe (36 mg, 0.67 mmol). The solution was stirred at room temperature for 25 minutes after which time TLC (20% MeOH in EtOAc) indicated consumption of the starting material and formation of a single product (Rf=0.3). The reaction mixture was quenched by the addition of DOWEX-50WX8 (H+form) until the pH was neutral (pH paper). The solution was filtered and rinsed with MeOH. The solvent was evaporated to give the title compound as a thick clear oil (141 mg, 95%). 1H NMR (400 MHz, CDCl3): δH=3.54 (1H, ddd, J=2.3, 5.6, 9.4), 3.64 (1H, t, J=9.6), 3.71-3.75 (2H, m, H3 and H6), 3.83-3.86 (2H, m, H2 and H6′), 3.99-4.04 (1H, m, CHH—CH═CH2), 4.20-4.25 (1H, m, CHH—CH═CH2), 4.81 (1H, d, J=1.5, H1), 5.18 (1H, dd, J=10.4, 1.8, CH═CHH), 5.31 (1H, dd J=17.2, 1.8, CH═CHH), 5.90-5.99 (1H, m, CH═CH₂).

### Example 13

### Preparation of MSH

### Part A: Ethyl-O-(mesitylenesulfonyl)acetohydroxamate (MSH Precursor)

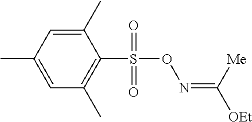

Ethyl N-hydroxyacetimidate (2.36 g, 22 9 mmol) was dissolved in DMF (6 mL). Triethylamine (3 mL) was added and the stirred solution was cooled to 0° C. 2-Mesitylenesulfonyl chloride (5.00 g, 22 9 mmol) was added in two portions and the resulting white slurry was stirred vigorously for 15 min. The mixture was then diluted with CH₂Cl₂ (100 mL) and washed repeatedly with H₂O. The organic layer was dried (MgSO₄), filtered, and the solvent removed under reduced pressure to give the title compound as a white solid that was used without further purification. m.p.=51-53° C. Spectroscopic data was identical to that previously reported (Tamura, Y.; Minamikawa, J.; Sumoto, K.; Fujii, S.; Ikeda, M., J. Org. Chem. 1973, 38, 1239-1241).

### Part B: O-Mesitylsulfonylhydroxylamine (MSH)

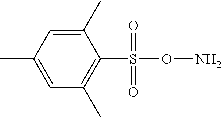

A solution of ethyl-O-(mesitylsulfonyl)acetohydroxamate (4.42 g, 15.49 mmol) in dioxane (4 mL) was cooled to 0° C. Perchloric acid (70%, 1.80 mL) was added dropwise by pipette over 2 minutes. After stirring for 5 minutes the mixture solidified. The contents of the reaction were transferred to 200 mL of ice water and the flask rinsed with H₂O (50 mL) and Et₂O (50 mL). The contents were transferred to a separatory funnel and extracted with Et₂O (40 mL). The organic layer was neutralized and partially dried with anhydrous potassium carbonate and then filtered. The filtrate was concentrated to less than 100 mL total volume and then poured into 150 mL of ice cold petrol and left to crystallize for 30 min. The white crystals (small needles) were isolated by filtration, transferred to a plastic Falcon tube, and dried under vacuum. The dried product (3.11 g, 93% yield) was stored at −20° C. and sealed with no more than wax film. m.p.=90-91° C.; ¹H NMR (400 MHz, CDCl₃): δ_{H}=2.32 (3H, s, CH₃Ar), 2.63 (6H, s, 2×CH₃Ar), 6.58 (2H, br s, NH₂), 6.98 (2H, s, Ar—H).

### Example 14

### Allyl Thioacetate

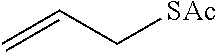

To a 500 mL round bottom flask equipped with a Teflon coated stir bar was added DMF (70 mL), K₂CO₃ (24.87 g, 180 mmol), and allyl chloride (20.0 mL, 245 mmol). The flask was equipped with a rubber septum and placed under argon. The stirred mixture was cooled to 0° C. and thioacetic acid (11.64 mL, 164 mmol) was added slowly. An efficient exit through a bubbler was ensured to compensate for CO₂ evolution. After gas evolution had subsided, the ice bath was removed and the reaction stirred vigorously at room temperature for 1.5 hours. TLC (5% EtOAc in petrol) indicated consumption of thioacetic acid (R_{f}=0.0 to 0.1) and formation of allyl thioacetate (R_{f}=0.7). The reaction mixture was transferred to a separatory funnel and diluted with Et₂O (600 mL) and H₂O (250 mL). The layers were separated and the organic layer was washed successively with NaHCO₃ (sat. aq., 250 mL), H₂O (250 mL), and brine (250 mL). The organic layer was dried (MgSO₄) and filtered. The solution was concentrated to ˜100 mL by rotary evaporation at a pressure of 300 mm Hg with care taken not to exceed 25° C. The resulting yellow solution was purified by distillation (b.p.=54° C., 38 mm Hg) to give the title compound as a clear liquid (17.98 g, 95%). ¹H NMR (400 MHz, CDCl₃): δ_{H}=2.33 (3H, s, CH₃), 3.53 (2H, dd, J=7.1, 1.3, CH₂SAc), 5.09 (1H, dt, J=9.9, 1.3, HC═CHH cis), 5.23 (1H, dt, J=16.9, 1.3, HC═CHH trans), 5.79 (1H, m, HC═CH₂).

### Example 15

### Allylthiol (˜1M in MeOH/MeOAc)

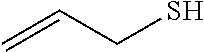

Allyl thioacetate, (16.62 g, 143 mmol) was added to a 250 round bottom flask equipped with a Teflon coated stir bar. Anhydrous methanol (80 mL) was added and the stirred solution was cooled to 0° C. Sodium methoxide (8.11 g, 150.2 mmol) was added in four portions over 10 min. After 10 minutes of stirring, TLC (30% EtOAc in petrol) revealed that all thioester had been consumed, with all product at the baseline. The reaction was quenched with DOWEX® 50WX8 (H⁺ form) until the pH was ˜7, as indicated by pH paper. The reaction was filtered and then distilled directly (b.p.=67° C., 760 mm Hg) to give the title compound as a solution in MeOH and MeOAc (MeOH and MeOAc co-distilled with allyl thioacetate). Assuming a density of MeOH (˜0.8 g/mL), the concentration of allylthiol is ˜1.08 M, as judged by ¹H NMR. Yield=125 mL=135 mmol=94%. ¹H NMR (400 MHz, CDCl₃): δ_{H}=1.41 (1H, t, J=7.8, SH), 3.13 (2H, t, J=7.8, CH₂SH), 4.97 (1H, ad, J=10.1, CH═CHH cis), 5.12 (1H, dd, J=16.8, 1.4, CH═CHH trans), 5.90 (1H, m, CH═CH₂). This solution was used directly in subsequent reactions.

### Example 16

### SBL-156-S-Allyl-Cysteine (SBL-156Sac)

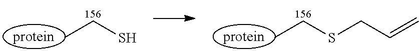

SBL-S156C is a single cysteine mutant of the serine protease subtilisin Bacillus lentus (SBL) which contains a cysteine residue at the 156 position in its amino acid sequence. SBL-S156C was prepared as a 1.4 mg/mL solution in 50 mM sodium phosphate buffer (pH 8.0) and 1.00 mL (0.052 μmol) was added to a 1.50 mL plastic tube. MSH (1.2 mg, 5.6 μmol) was added as a solution in DMF (50 μL) and the reaction vortexed immediately upon addition. The homogenized sample was shaken at 4° C. for 20 minutes before a 40 μL aliquot was analyzed by LC-MS, confirming full conversion of Cys156 to Dhal56 (26681 calculated mass, 26681 found). Allylthiol (190 μL of the 1.08 M solution in MeOH prepared in Example 10) was added directly to the protein mixture and vortexed to homogenize. The reaction was rotated at room temperature for 30 minutes and then analyzed directly by LC-MS, confirming full conversion to S-allyl-cysteine (Sac). (26755 calculated mass, 26758 found). Small molecules were removed with a PD10 column (GE Healthcare), eluting with 50 mM sodium phosphate buffer (pH 8.0). The sample was further purified by dialysis (2× against 4L 50 mM sodium phosphate, pH 8.0). The sample was then split into 350 μL aliquots, flash frozen, and stored at −80° C. The final concentration of product was 0.4 mg/mL, measured by Bradford's method (Bradford, M. Anal. Biochem. 1976, 72, 248-254).

### Example 17

### Cross Metathesis on SBL-S156Sac With Allyl Alcohol

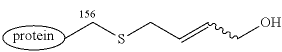

All manipulations were carried out at room temperature. SBL-C156Sac was thawed and stored on ice until needed (previously prepared as described in Example 16, 0.4 mg/mL in 50 mM sodium phosphate, pH 8.0). A saturated solution of Grubbs-Hoveyda catalyst V was prepared by repeatedly vortexing and sonicating 0.8 mg of the catalyst in 115 μL ^{t}BuOH. A 250 μL aliquot of SBL-C156Sac (0.004 μmol) was transferred to a 1.5 mL plastic tube. MgCl₂.6H₂O (7.1 mg, 35 μmol) and ^{t}BuOH (36 μL) were both added to the protein solution and the sample vortexed to homogenize. An aliquot of the Grubbs-Hoyveda catalyst solution was then added (71.5 μL, ˜0.75 μmol) and the sample vortexed. Finally, allyl alcohol (2.5 μL, 37 μmol) was added. The reaction tube was rotated on a lab rotisserie at room temperature. The progress of the reaction was monitored directly by LC-MS. After 5 hours of reaction time, >90% conversion to cross metathesis product was observed.

### Example 18

### Cross Metathesis on SBL-S156Sac With Allyl Glucoside

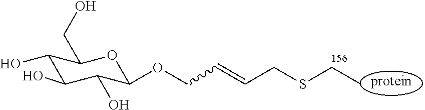

All manipulations were carried out at room temperature. SBL-C156Sac was thawed and stored on ice until needed (previously prepared as described in Example 16, 0.4 mg/mL in 50 mM sodium phosphate, pH 8.0). A saturated solution of Grubbs-Hoyveda catalyst V was prepared by repeatedly vortexing and sonicating 0.9 mg of the catalyst in 141 μL ^{t}BuOH. A 250 μL aliquot of SBL-C156Sac (0.004 μmol) was transferred to a 1.5 mL plastic tube. MgCl₂.6H₂O (6.5 mg, 30 μmol) and ^{t}BuOH (36 μL) were both added to the protein solution and the sample vortexed to homogenize. An aliquot of the Grubbs-Hoyveda catalyst solution was then added (71.5 μL, ˜0.73 μmol) and the sample vortexed. Finally, allyl glucoside (2.5 mg, 11 μmol) was added and the reaction vortexed and then placed on rotating wheel at room temperature. The progress of the reaction was monitored by LC-MS. After 1 hour at room temperature, no product formation was observed so the reaction tube was incubated at 37° C. After 4 hours at 37° C., 50% conversion of to cross metathesis product was observed.

### Example 19

### Cross Metathesis of SBL-S156Sac With Allyl Mannoside

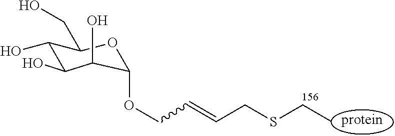

All manipulations were carried out at room temperature. SBL-C156Sac was thawed and stored on ice until needed (previously prepared as described in Example 16, 0.57 mg/mL in 50 mM sodium phosphate, pH 8.0). A saturated solution of Grubbs-Hoveyda II was prepared by repeatedly vortexing and sonicating 0.83 mg of the catalyst in 100 μL ^{t}BuOH. A 250 μL aliquot of SBL-C156Sac (0.0053 μmol) was transferred to a 1.5 mL plastic tube. MgCl₂.6H₂O (10.8 mg, 53 μmol) and ^{t}BuOH (36 μL) were both added to the protein solution and the sample vortexed to homogenize. An aliquot of the Grubbs-Hoveyda catalyst solution was then added (71.5 μL, ˜1.1 μmol) and the sample vortexed. Finally, allyl mannoside (11.7 mg, 53 μmol) was added and the reaction vortexed and then placed on rotating wheel at room temperature. The progress of the reaction was monitored by LC-MS. After 1 hour at room temperature, the reaction tube was incubated at 37° C. After 4 hours at 37° C., 60% conversion of to cross metathesis product was observed (Calculated mass: 26947, observed mass: 26951).

### Example 20

### SBL Activity After Metathesis Reaction With Allyl Alcohol

An aliquot of the reaction mixture of the cross-metathesis product of Example 17 was prepared at a concentration of 0.1 mg/mL in pH 8.0 sodium phosphate buffer (50 mM) and 250 μL was added to 500 μL plastic tube. A 25 μL aliquot of the peptide SucAAPFpNA (0.20 M in DMSO, Bachem) was added and the reaction mixture turned bright yellow immediately upon addition. The resulting yellow solution indicated liberation of p-nitroanaline and verified peptidase activity of the modified SBL The modified protein and sucAAPFpNA alone are clear solutions at the same concentration.

### Example 21

### S-Allylcysteine (Sac)

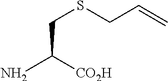

BocSacOMe (9.92 g, 36.02 mmol) was added to a 250 round bottom flask and dissolved in CH₂Cl₂ (100 mL). The solution was placed under argon and TFA (10 mL) was added at room temperature. The reaction was stirred at room temperature for 3 hours after which time TLC (15% EtOAc in petrol) indicated consumption of starting material (R_{f}=0.4). The solvent was then removed by rotary evaporation and the resulting yellow residue was dried briefly under vacuum. The viscous residue was then dissolved in THF (30 mL) and the stirred solution was cooled to 0° C. LiOH (72 mL of a 5M solution) was then poured into the reaction mixture. The ice bath was removed and the reaction stirred at room temperature for 1 hour after which time the reaction was diluted with 100 mL H₂O and then neutralized (pH<7, pH paper) with DOWEX® 50WX8 (H⁺, ˜120 g). All of the resin was poured into an empty column and washed with 500 mL of H₂O (gravity flow). The flow-through was discarded. The product was eluted with 5% NH₄OH (aq) and the fractions containing the title compound were collected [R_{f}=0.6; 7:2:1 ⁱPrOH:MeOH:NH₄OH (25% aq)]. The product was concentrated by rotary evaporation to give a yellow solid which was then purified by column chromatography [7:2:1 ⁱPrOH:MeOH:NH₄OH (25% aq.)] to give Sac as white crystals (3.29 g, 57%). m.p.=212-213° C. ¹H NMR (400 MHz, D₂O): δ_{H}=2.5-2.70 (2H, ABX system, J=13.3, 5.3, 6.7, CH₂SAllyl), 3.06 (2H, d, J=7.2, CH₂CH═CH₂), 3.28 (1H, dd, J=6.7, 5.3, H_{α}), 5.02-5.09 (2H, m, HC═CH₂), 5.71 (1H, m, HC═CH₂).

### Example 22

### Expression of Single Met Sulfolobus solfataricus β-glycosidase (SsβG) Mutant With Sac as a Met Surrogate

Sequence for single methionine construct of Sulfolobus solfataricus β-glycosidase (SsβG) M21I M73I M148I M206I M236I M275I M280I C344S M383I M439I with N-terminally fused His₇ tag GHHHHHHH. Single methionine (intended site of Sac incorporation) is denoted as “X” below (PDB code=1GOW for wild type) (Aguilar, C. F.; Sanderson, I.; Moracci, M.; Ciaramella, M.; Nucci, R.; Rossi, M.; Pearl, L. H. J. Mol. Biol. 1997, 271, 789-802).

GHHHHHHHSFPNSFRFGWSQAGFQSEIGTPGSEDPNT
DWYKWVHDPENXAAGLVSGDLPENGPGYWGNYKTFHDNAQKIGLKIARLN
VEWSRIFPNPLPRPQNFDESKQDVTEVEINENELKRLDEYANKDALNHYR
EIFKDLKSRGLYFILNIYHWPLPLWLHDPIRVRRGDFTGPSGWLSTRTVY
EFARFSAYIAWKFDDLVDEYSTINEPNVVGGLGYVGVKSGFPPGYLSFEL
SRRAIYNIIQAHARAYDGIKSVSKKPVGIIYANSSFQPLTDKDIEAVEIA
ENDNRWWFFDAIIRGEITRGNEKIVRDDLKGRLDWIGVNYYTRTVVKRTE
KGYVSLGGYGHGSERNSVSLAGLPTSDFGWEFFPEGLYDVLTKYWNRYHL
YIYVTENGIADDADYQRPYYLVSHVYQVHRAINSGADVRGYLHWSLADNY
EWASGFSIRFGLLKVDYNTKRLYWRPSALVYREIATNGAITDEIEHLNSV
PPVKPLRH

Calculated average isotopic mass (N-terminal Met cleaved)=57235.8 (if X=Met); 57247.8 (if X=Sac)

Incorporation of S-allylcysteine Into Single Met Mutant of SsβG.

Four expression experiments were carried out in parallel, designated as follows:
A=S-allylcysteine final conc.=40 μg mL⁻¹
B=S-allylcysteine final conc.=1000 μg mL⁻¹
C=control (1 wash), S-allylcysteine final conc.=0 μg mL⁻¹
D=control (2 washes), S-allylcysteine final conc.=0 μg mL⁻¹

An overnight culture of Escherichia coli B834 (DE3), pET24d M21I M73I M148I M206I M236I M275I M280I C344S M383I M439I SsrβG was grown in SelenoMet™ media (200 mL) (Molecular Dimensions) supplemented with kanamycin (final conc. 50 μg mL⁻¹) and L-methionine (final conc. 40 μg mL⁻¹) (>16 hours). The overnight culture was used to inoculate pre-warmed (37° C.) SelenoMet™ media (2.25 L (A), 1.5 L (B, C), 0.75 L (D)) supplemented with kanamycin (final conc. 50 μg mL⁻¹) and L-methionine (final conc. 40 μg mL⁻¹). The cells were incubated (37° C., 220 rpm). The optical density (OD₆₀₀) was monitored using a blank as a reference until a value of <1.0 was obtained (ca. 3 hours). The medium shift was performed by centrifugation (8000 rpm, 8 minutes, 4° C.), washing with SelenoMet™ media (400 mL) once (A, C) or twice (B, D), and transfer to pre-warmed (37° C.) SelenoMet™ media (2.25 L (A), 1.5 L (B, C), 0.75 L (D)) supplemented with kanamycin (final conc. 50 μg mL⁻¹) and S-allylcysteine (final conc. 40 μg mL⁻¹ (A), final conc. 1000 μg mL⁻¹ (B), final conc. 0 μg mL⁻¹ (C, D)). The cultures were incubated (30 minutes at 37° C. then 30 minutes at 30° C., 220 rpm) before induction by addition of IPTG (final conc. 1.0 mM). Expression was continued for 12 hours (30° C., 220 rpm). The cells were harvested by centrifugation (8000 rpm, 8 minutes, 4° C.) and were stored in binding buffer (0.2 mM Tris, 5 mM imidazole, 0.5 M NaCl, pH 7.8) (30 mL) at −20° C. The cells were thawed; DNase (10 μL) and lysozyme (10 mg) were added and the cell suspension stirred for 30 minutes. The cells were lysed by sonication, harvested by centrifugation (20 minutes, 18000 rpm, 4° C.) and the lysate sterile filtered (0.4 μm). Protein purification was accomplished using an AKTA FPLC system and HisTrap™ FF 1 mL or 5 mL column. The column was charged with NiSO₄ (0.1 M) and washed with binding buffer before use. The protein was injected and eluted at 1.0 mL min⁻¹ using a gradient system from 100% binding buffer to 100% elution buffer (0.2 mM Tris, 300 mM imidazole, 0.5 M NaCl, pH 7.8). All buffers were filtered and degassed before use. All eluent collected from purification was analysed by SDS PAGE (shown below) and that shown to contain pure protein was combined and dialysed against 50 mM phosphate buffer (pH 6.5) (A) or buffer exchanged into 50 mM phosphate buffer (pH 6.5) using a PD10 column (B, C, D). β-galactosidase activity was measured qualitatively; X-Gal solution (100 μL) was added to protein sample (50 μL) and the mixture was incubated at 37° C. for 1 hour. A colour change from yellow to blue indicated β-galactosidase activity for all expressed proteins (FIG. S6). ESI-MS was obtained. For all four purified proteins from cultures A, B, C, and D, total mass ESI-MS indicated methionine incorporation (57236 calculated, 57236 found). No Sac incorporation was detected by total mass ESI. Control incorporation of Met suggests residual methionine is not completely removed during washes. To ascertain if any of the expressed protein contained Sac, the purified protein was digested and analyzed by MS-MS.

### Example 23

### Tryptic Digest of Expressed Protein in Attempted Sac Incorporation Into Single Met SsβG

An SDS PAGE gel (NuPAGE 4-12% Bis-Tris, Invitrogen) of protein sample A was run (see above). A clean razor blade was used to excise the band corresponding to the expressed protein. The gel slice was placed into a microcentrifuge tube and was cut into ˜1 mm³ pieces. The gel slices were washed sequentially as follows and rotated on a lab rotisserie: a) water/acetonitrile 50:50 (100 μL, 30 mins), b) 0.1 M ammonium bicarbonate/acetonitrile 50:50 (100 μL, 30 mins), c) water (100 μL, 15 mins), d) 0.1 M ammonium bicarbonate/acetonitrile 50:50 (100 μL, 30 mins), e) water (100 μL, 15 mins), f) 0.1 M ammonium bicarbonate/acetonitrile 50:50 (100 μL, 30 mins), g) water (100 μL, 5 mins). The liquid was removed and the gel slices were dehydrated using 100% acetonitrile (50 μL). When the gel slices were white and coagulated (˜5 minutes), the acetonitrile was removed and the gel slices were rehydrated using 0.1 M ammonium bicarbonate (50 μL). After 5 minutes, acetonitrile (50 μL) was added. The gel slices were left for 15 minutes without shaking before drying in a Speed Vac for 1 hour at room temperature. Trypsin (20 μg) (Promega) was suspended in 50 mM ammonium bicarbonate (1 mL). The gel slices were rehydrated in trypsin solution (50 μL) and incubated at 37° C. overnight. The liquid was transferred to a microcentrifuge tube. 25 mM ammonium bicarbonate (100 μL) was added. After 5 minutes of shaking, acetonitrile (100 μL) was added. The gel slices were shaken for 1 hour. The liquid was transferred to the microcentrifuge tube and 0.1% formic acid (100 μL) was added. After 5 minutes of shaking, acetonitrile (100 μL) was added. The gel slices were shaken for 1 hour. The liquid was transferred to the microcentrifuge tube. The combined liquids were freeze dried and resuspended in 0.1% formic acid (10 μL). The tryptic samples was analysed by liquid chromatography (Agilent) using a Phenomenex Jupiter 5u C18 300A 150×0.5 mm column coupled to an ESI-TOF MS (Q-Tof micro™ Micromass). The tryptic peptides were injected and eluted at 15 μL min⁻¹ using a 90 min gradient system, using solvent A (water/0.1% formic acid) and solvent B (acetonitrile/0.1% formic acid). The output of the liquid chromatography was injected into the mass spectrometer with a scan range of 200-2800 m/z, capillary voltage 3000 V, cone voltage of 35 V, source temperature of 80° C., collision energy of 35 V and desolvation temperature of 200° C.

The predicted peptide fragment containing Met43 (Cut at Lys35 and Lys64) is highlighted below:

GHHHHHHHSFPNSFRFGWSQAGFQSEIGTPGSEDPNTDWYKWVHDPENXA
AGLVSGDLPENGPGYWGNYKTFHDNAQKIGLKIARLNVEWSRIFPNPLPR
PQNFDESKQDVTEVEINENELKRLDEYANKDALNHYREIFKDLKSRGLYF
ILNIYHWPLPLWLHDPIRVRRGDFTGPSGWLSTRTVYEFARFSAYIAWKF
DDLVDEYSTINEPNVVGGLGYVGVKSGFPPGYLSFELSRRAIYNIIQAHA
RAYDGIKSVSKKPVGIIYANSSFQPLTDKDIEAVEIAENDNRWWFFDAII
RGEITRGNEKIVRDDLKGRLDWIGVNYYTRTVVKRTEKGYVSLGGYGHGS
ERNSVSLAGLPTSDFGWEFFPEGLYDVLTKYWNRYHLYIYVTENGIADDA
DYQRPYYLVSHVYQVHRAINSGADVRGYLHWSLADNYEWASGFSIRFGLL
KVDYNTKRLYWRPSALVYREIATNGAITDEIEHLNSVPPVKPLRH
WVHDPENXAAGLVSGDLPENGPGYWGNYK

Calculated average isotopic mass: 3174.4 (if X=Met); 3186.4 (if X=Sac).

Found: [M+3H]³⁺=1059.2 (Met incorporation, m/z=1059.1 calculated for [M+3H]³⁺)

Retention time=28.99 min.

Found: [M+3H]³⁺=1063.2 (Sac incorporation, m/z=1063.1 calculated for [M+3H]³⁺)

Retention time=29.33 min.

The site and identity of X was determined unambiguously by MS-MS analysis of the peptide fragments observed above.

### Example 24

### Allyl methyl triethylene glycol

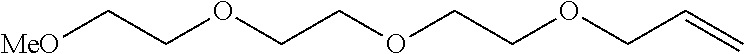

To a flame dried 100 mL three-neck round bottom flask under nitrogen was added DMF (40 mL, anhydrous). Sodium hydride (306 mg, 60% wt. in mineral oil, 7.66 mmol) was added under a stream of nitrogen. The stirred suspension was cooled to 0° C. and triethylene glycol monomethyl ether (1.00 mL, 6.38 mmol) was added dropwise and then stirred for 5 minutes upon completion of addition. Allyl chloride (1.31 mL, 15.95 mmol) was then added and the reaction was stirred at 0° C. for 40 minutes before quenching carefully with 20 mL H₂O. The mixture was diluted with 300 mL EtOAc and washed successively with H₂O (2×150 mL) and brine (200 mL). The organic layer was dried (Na₂SO₄), filtered, and concentrated under reduced pressure. The resulting residue was purified by column chromatography (EtOAc) to give the title compound as a clear oil (778 mg, 60%). ¹H NMR (400 MHz, CD₃OD): δ_{H}=3.38 (3H, s, OCH₃), 3.54-3.57 (2H, m, CH₂OMe), 3.60-3.67 (10H, m, —OCH₂CH₂O—), 4.03 (2H, dt, J=1.4, 5.5, OCH₂CH═CH₂), 5.16-5.20 (1H, m, CH═CHH), 5.27-5.33 (1H, m, CH═CHH), 5.89-5.97 (1H, m, CH═CH₂).

### Example 25

### Tetraethylene glycol mono(tert-butyl dimethylsilyl)ether

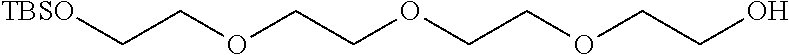

CH₂Cl₂ (100 mL) was added to a 250 mL round bottom flask and flushed with nitrogen. Tetraethylene glycol (4.00 mL, 23.17 mmol) and imidazole (4.73 g, 69.51 mmol) were added and stirred to dissolve. The stirred solution was cooled to 0° C. and tert-butyl dimethylsilyl chloride (TBSCl, 4.32 g, 27.80 mmol) was added in a single portion. After stirring for 40 min at 0° C., TLC (EtOAc) revealed the di-silylated product (R_{f}=0.6) and the mono-silyl ether (R_{f}=0.3). The reaction was quenched with 250 mL of H₂O and the organic layer separated. The aqueous layer was then extracted with CH₂Cl₂ (2×100 mL). The combined organics were washed with brine (200 mL), dried (Na₂SO₄), filtered, and concentrated under reduced pressure. Purification by column chromatography (EtOAc) afforded the title compound as a clear oil (2.92 g, 41%). ¹H NMR (400 MHz, CDCl₃): δ_{H}=0.01 (6H, s, TBS), 0.83 (9H, s, TBS), 2.97 (1H, br. s, OH), 3.48-3.72 (16H, m, —OCH₂CH₂O—).

### Example 26

### Allyl(dimethyl-tert-butyl silyl)tetraethyleneglycol

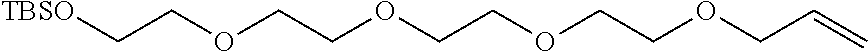

To a flame dried 250 mL 2-neck round bottom flask under nitrogen was added DMF (40 mL, anhydrous). Sodium hydride (311 mg, 60% wt. in mineral oil, 7.78 mmol) was added under a stream of nitrogen and the stirred suspension was cooled to 0° C. Tetraethylene glycol mono(tert-butyl dimethylsilyl)ether (2.00 g, 6.48 mmol) was added dropwise to the reaction and then stirred at 0° C. for 3 min. Allyl chloride (1.33 mL, 16.20 mmol) was then added and the reaction stirred at 0° C. for 40 minutes. The reaction was then quenched at 0° C. by slow addition of H₂O (10 mL). The mixture was diluted with EtOAc (250 mL) and H₂O (250 mL). The organic layer was separated, washed successively with H₂O (2×250 mL) and brine (150 mL), dried (Na₂SO₄), and filtered. After concentrating under reduced pressure, the resulting residue was purified by column chromatography (50% EtOAc in petrol) to afford the title compound as a clear oil (777 mg, 34%). ¹H NMR (400 MHz, CDCl₃): δ_{H}=0.04 (6H, s, TBS), 0.87 (9H, s, TBS), 3.53 (2H, t, J=3.57-3.59 (2H, m), 3.61-3.65 (10H, m), 3.74 (2H, t, J=5.4) (—OCH₂CH₂O—) (2H, dt, J=1.4, 5.6, CH₂CH═CH₂), 5.13-5.17 (1H, m, CH═CHH), 5.21-5.28 (1H, m, CH═CHH), 5.84-5.94 (1H, m, CH═CH₂).

### Example 27

### Tetraethylene glycol monoallyl ether

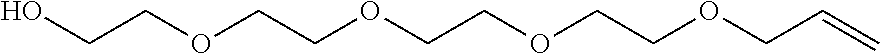

Allyl(dimethyl-tert-butyl silyl)tetraethyleneglycol (275 mg, 0.79 mmol) was added to a 50 mL round bottom flask and flushed with argon before dissolving in THF (10 mL, anhydrous). The stirred solution was cooled to 0° C. and TBAF (1.18 mL of a 1.0 M solution in THF) was added dropwise. The reaction was stirred for 30 min and another portion of TBAF (1.0 mL of a 1.0 M solution in THF) was added. After 10 minutes, TLC (10% MeOH in EtOAc) revealed complete consumption of the starting material (R_{f}=0.7) and formation of the product (R_{f}=0.3). The reaction was quenched by the addition of DOWEX 50WX8 (H⁺). The resin was filtered off and rinsed with MeOH. The filtrate was concentrated and then purified by column chromatography (10% MeOH in EtOAc) to afford the title compound as a clear oil (174 mg, 94%). ¹H NMR (400 MHz, CDCl₃): δ_{H}=3.56-3.68 (16H, m, —OCH₂CH₂O—), 4.03 (2H, dt, J=1.5, 5.6, CH₂CH═CH₂), 5.15-5.20 CH═CHH), 5.27-5.33 (1H, m, CH═CHH), 5.88-5.98 (1H, m, CH═CH₂).

### Example 28

### Cross Metathesis on SBL-S156Sac With allyl methyl triethylene glycol

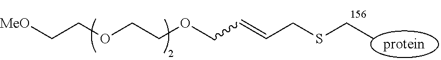

All manipulations were carried out at room temperature. SBL-C156Sac was thawed and stored on ice until needed (previously prepared as described in Example 16, 0.57 mg/mL in 50 mM sodium phosphate, pH 8.0). A saturated solution of Grubbs-Hoveyda II was prepared by repeatedly vortexing and sonicating 2.0 mg of the catalyst in 330 μL ^{t}BuOH. A 250 μL aliquot of SBL-C156Sac (0.005 μmol) was transferred to a 1.5 mL plastic tube. MgCl₂.6H₂O (11.7 mg, 57 μmol) and ^{t}BuOH (36 μL) were both added to the protein solution and the sample vortexed to homogenize. An aliquot of the Grubbs-Hoveyda catalyst solution was then added (71.5 μL, ˜1.1 μmol) and the sample vortexed. Finally, allyl methyl triethylene glycol (5.4 mg, 27 μmol) was added and the reaction, vortexed, and placed on rotating wheel at room temperature. The progress of the reaction was monitored by LC-MS. After 2 hour at room temperature, 20% product formation was observed. The reaction tube was then incubated at 37° C. After 1 hour at 37° C., 55% conversion of to cross metathesis product was observed. Both the product (calculated mass: 26931, observed mass: 26926) and the magnesium adduct thereof were observed (calculated mass: 26955, observed mass: 26953).

### Example 29

### Cross Metathesis on SBL-S156Sac with Allyl tetraethylene glycol

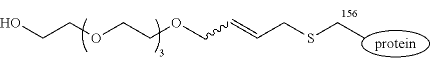

All manipulations were carried out at room temperature. SBL-C156Sac was thawed and stored on ice until needed (previously prepared as described in Example 16, 0.57 mg/mL in 50 mM sodium phosphate, pH 8.0). A saturated solution of Grubbs-Hoveyda II was prepared by repeatedly vortexing and sonicating 2.0 mg of the catalyst in 330 μL ^{t}BuOH. A 250 μL aliquot of SBL-C156Sac (0.005 μmol) was transferred to a 1.5 mL plastic tube. MgCl₂.6H₂O (11.0 mg, 54 μmol) and ^{t}BuOH (36 μL) were both added to the protein solution and the sample vortexed to homogenize. An aliquot of the Grubbs-Hoveyda catalyst solution was then added (71.5 μL, ˜1.1 μmol) and the sample vortexed. A stock solution of tetraethylene glycol monoallyl ether was prepared by dissolving 18.6 mg in 50 μL of 30% tBuOH in 50 mM sodium phosphate (pH 8.0). A 16.8 μL aliquot of this solution (6.2 mg, 27 μmol) was added to the reaction and the tube was vortexed and placed on rotating wheel at room temperature for 2 hours and then 1 hour at 37° C. LC-MS analysis revealed 60% conversion of to cross metathesis product. Both the product (calculated mass: 26961, observed mass: 26955) and the magnesium adduct thereof (calculated mass: 26985, observed mass: 26983) were observed.

### Example 30

### Allyl Selenocyanate

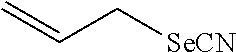

KSeCN (3.27 g, 22.72 mmol) was added to a 100 mL round bottom flask and dissolved in DMF (25 mL). The solution was placed under an atmosphere of nitrogen and allyl chloride (3.72 mL, 45.43 mmol) was added slowly to the stirred solution. The reaction was stirred for 20 minutes at room temperature and then diluted with Et₂O (200 mL) and washed sequentially with H₂O (2×200 mL) and brine (200 mL). The organic layer was dried (MgSO₄), filtered, and concentrated under reduced pressure. The product was isolated as a pale yellow liquid and was sufficiently pure to use in subsequent manipulations (1.25 g, 38%).

### Example 31

### Allylation of SBL-S156C With Allyl Chloride

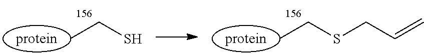

Method A: Direct Allylation

SBL-S156C (2.5 mL, 1 mg/mL, pH 8.0 sodium phosphate, 94 nmol) was added to a 15 mL Falcon tube and stored on ice until needed. Allyl chloride was prepared as a 0.65 M solution in DMF. A 147 μL, aliquot of the allyl chloride solution (96 nmol) was added to the protein solution and the reaction was vortexed immediately upon addition. The reaction was incubated at 37° C. for 30 minutes and then analyzed directly by LC-MS, confirming full conversion to the allylated product SBL-156-Sac (26755 calculated mass, found 26753). Small molecules were removed with a PD10 column (GE Healthcare), eluting with 3.5 mL 50 mM sodium phosphate buffer (pH 8.0). The sample was then split into 200 μL aliquots, flash frozen, and stored at −20° C. The absence of free thiol groups in the product was confirmed using Ellman's test.

### Method B: Pre-Reduction With DTT

SBL-S156C (2.5 mL, 1.0 mg/mL, pH 8.0 sodium phosphate, 94 nmol) was added to a 15 mL Falcon tube and stored on ice. Dithiothreitol (DTT) (3.6 mg, 23 μmol) was added as a solid to reduce any contaminant disulfide. The solution was vortexed and then shaken for 10 minutes at room temperature. Allyl chloride (19 μL, 230 nmol) was then added as a solution in DMF (200 μL). The mixture was vortexed and then shaken at 37° C. for 30 min. LC-MS analysis revealed full conversion to the allylated product. (26755=calculated mass, found 26756). The reaction mixture was passed through a PD10 column previously equilibrated with pH 8.0 sodium phosphate (50 mM). The product was split into 200 μL aliquots and flash frozen.

### Example 32

### Olefin metathesis on SBL-156Sac from allyl chloride allylation

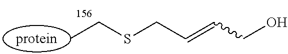

Olefin metathesis was carried out by a procedure analogous to that of Example 17. LC-MS after 1 hour of reaction time revealed formation of product. Full conversion to the product was observed after 2 hours (26785 calculated mass, found 26786).

### Example 33

### Allylation of SBL-S156C With Allyl Selenocyanate

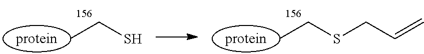

SBL-S156C (200 μL, 1.0 mg/mL, pH 8.0 sodium phosphate, 7.5 nmol) was added to a 1.0 mL plastic tube and stored on ice. A stock solution of allyl selenocyanate (11.9 mg) was prepared in DMF (541 μL). A 5 μL aliquot of the selenide solution (0.75 μmol) was added to the protein sample. The reaction was shaken for 10 minutes at room temperature and a 30 μL aliquot was analyzed by LC-MS. A mixture of selenenyl sulfide and allylcysteine was observed (˜5:4). (26834 calculated for selenenyl sulfide; 26755 calculated for allylsulfide). After 1 hour of total reaction time, the reaction was analyzed by LC-MS. Full conversion to the allyl sulfide was observed. (26755=calculated mass; 26755 found). Small molecules were removed by passing the sample through a PD10 column previously equilibrated with pH 8.0 sodium phosphate (50 mM). The absence of free thiol groups in the product was confirmed using Ellman's test. No difference in cross-metathesis reactivity with allyl alcohol was observed between the product of this Example and the product of Example 31A.

### Example 34

### Peptidase Activity Assay of Allylated SBL-S156C

SBL-S156C (unmodified), SBL-156Sac (from Example 31A), and SBL-156Sac (from Example 33) were prepared at a concentration of 0.1 mg/mL in pH 8.0 sodium phosphate (50 mM). 225 μL aliquots of each sample were added to a 96-well plate. A 25 μL aliquot of SucAAPFpNA (0.20 M in DMSO, Bachem) was added to each of the protein samples. All reactions turned yellow immediately upon addition of the peptide substrate. The yellow solution indicates liberation of p-nitroaniline (pNA), confirming peptidase activity of all samples. The allylation reactions of Examples 31A and 33 did not therefore cause loss of protein activity.

What is claimed is:

1. A method for the modification of an amino acid, protein or peptide, the method comprising reacting a carbon-carbon double bond-containing compound I with an amino acid, a protein or a peptide containing an allyl sulfide group II in the presence of a catalyst which promotes olefin metathesis, to form a modified amino acid, protein or peptide III.

2. A method according to claim 1, wherein the carbon-carbon double bond-containing compound I is a compound 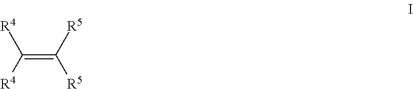
wherein
each R⁴ independently denotes H or C₁₋₁₀ alkyl; and
each R⁵ independently denotes H or any organic moiety it is desired to introduce into an amino acid, peptide or protein.

3. A method according to claim 2, wherein at least one of the R⁵ groups is a carbohydrate moiety, a polyethylene glycol (PEG) chain, a farnesyl group, a label or a pharmaceutically active compound.

4. A method according to claims 1, wherein the compound of formula I is selected from:

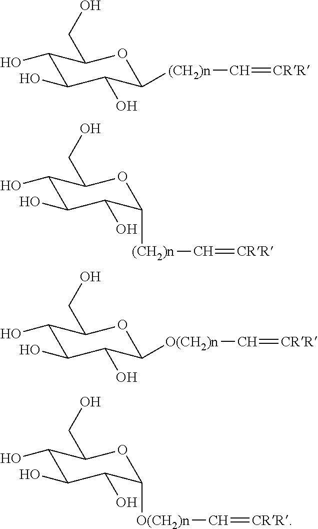

5. A method according to claim 1 wherein the amino acid, protein or peptide containing an allyl sulfide group II is a compound of formula 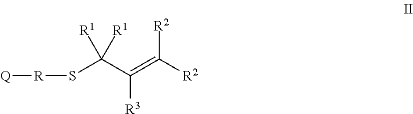
wherein
R denotes an amino acid side chain or a linker;
Q is an amino acid, peptide or protein;
each R¹ independently denotes H or C₁₋₁₀ alkyl;
each R² independently denotes H or C₁₋₁₀ alkyl; and
R³ denotes H or C₁₋₁₀ alkyl.

6. A method according to claim 1 wherein the modified amino acid, protein or peptide III is a compound of formula 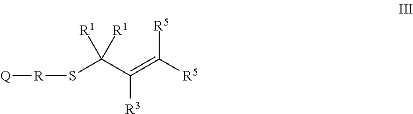
wherein
R denotes an amino acid side chain or a linker;
Q is an amino acid, peptide or protein;
each R¹ independently denotes H or C₁₋₁₀ alkyl;
R³ denotes H or C₁₋₁₀ alkyl; and
each R⁵ independently denotes H or an organic moiety.

7. A method according to claims 1, wherein the catalyst is a compound of formula V

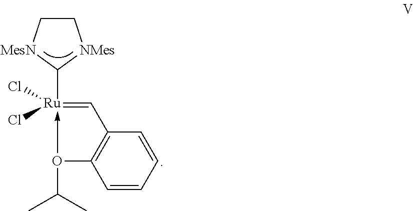

8. A method according to claim 1, wherein the reaction takes place in an aqueous solvent.

9. A method according to claim 1, wherein the amino acid is cysteine or the protein or peptide contains a cysteine residue.

10. A method according to claim 9 wherein the allyl sulfide group is prepared by conversion of cysteine or a cysteine residue to dehydroalanine and subsequent trapping with a thiol nucleophile.

11. A method according to claim 9 wherein the allyl sulfide group is prepared by reaction of cysteine or a cysteine residue with an allyl halide.

12. A method according to claim 9 wherein the allyl sulfide group is prepared by formation and dechalcogenative rearrangement of an allyl selenosulfide.

13. A method according to claim 9, wherein the allyl sulfide group is prepared by reacting cysteine or a cysteine residue with an compound of formula 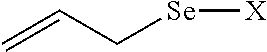
wherein X denotes a leaving group such as CN, SO₂Aryl (where Ar=e.g. phenyl or 4-methylphenyl), SO₂R (R=e.g. alkyl), SO₃⁻, I, Br, Cl, or OH, preferably CN.


SEQUENCE LISTING (5 sequences)

SEQ ID NO: 1 - protein, 8 aa, Artificial Sequence
  Description of Artificial Sequence; Note = Synthetic Construct
GHHHHHHH

SEQ ID NO: 2 - protein, 495 aa, Artificial Sequence
  Description of Artificial Sequence; Note = Synthetic Construct
GHHHHHHHSFPNSFRFGWSQAGFQSEIGTPGSEDPNTDWYKWVHDPENMAAGLVSGDLPENGPGYWGNYKTFHDNAQKIGLKIARLNVEWSRIFPNPLPRPQNFDESKQDVTEVEINENELKRLDEYANKDALNHYREIFKDLKSRGLYFILNIYHWPLPLWLHDPIRVRRGDFTGPSGWLSTRTVYEFARFSAYIAWKFDDLVDEYSTINEPNVVGGLGYVGVKSGFPPGYLSFELSRRAIYNIIQAHARAYDGIKSVSKKPVGIIYANSSFQPLTDKDIEAVEIAENDNRWWFFDAIIRGEITRGNEKIVRDDLKGRLDWIGVNYYTRTVVKRTEKGYVSLGGYGHGSERNSVSLAGLPTSDFGWEFFPEGLYDVLTKYWNRYHLYIYVTENGIADDADYQRPYYLVSHVYQVHRAINSGADVRGYLHWSLADNYEWASGFSIRFGLLKVDYNTKRLYWRPSALVYREIATNGAITDEIEHLNSVPPVKPLRH

SEQ ID NO: 3 - protein, 495 aa, Artificial Sequence
  Description of Artificial Sequence; Note = Synthetic Construct
GHHHHHHHSFPNSFRFGWSQAGFQSEIGTPGSEDPNTDWYKWVHDPENCAAGLVSGDLPENGPGYWGNYKTFHDNAQKIGLKIARLNVEWSRIFPNPLPRPQNFDESKQDVTEVEINENELKRLDEYANKDALNHYREIFKDLKSRGLYFILNIYHWPLPLWLHDPIRVRRGDFTGPSGWLSTRTVYEFARFSAYIAWKFDDLVDEYSTINEPNVVGGLGYVGVKSGFPPGYLSFELSRRAIYNIIQAHARAYDGIKSVSKKPVGIIYANSSFQPLTDKDIEAVEIAENDNRWWFFDAIIRGEITRGNEKIVRDDLKGRLDWIGVNYYTRTVVKRTEKGYVSLGGYGHGSERNSVSLAGLPTSDFGWEFFPEGLYDVLTKYWNRYHLYIYVTENGIADDADYQRPYYLVSHVYQVHRAINSGADVRGYLHWSLADNYEWASGFSIRFGLLKVDYNTKRLYWRPSALVYREIATNGAITDEIEHLNSVPPVKPLRH

SEQ ID NO: 4 - protein, 29 aa, Artificial Sequence
  Description of Artificial Sequence; Note = Synthetic Construct
WVHDPENMAAGLVSGDLPENGPGYWGNYK

SEQ ID NO: 5 - protein, 29 aa, Artificial Sequence
  Description of Artificial Sequence; Note = Synthetic Construct
WVHDPENCAAGLVSGDLPENGPGYWGNYK